(19) United States
(12) Patent Application Publication
(10) Pub. No.: US 2024/0063561 A1
(43) Pub. Date: Feb. 22, 2024

(54) MEDICAL DEVICE ASSEMBLY

(71) Applicant: Quanta Computer Inc., Taoyuan City (TW)

(72) Inventors: Jung-Wen CHANG, Taoyuan City (TW); Chien-Hung LIN, Taoyuan City (TW)

(21) Appl. No.: 18/050,601

(22) Filed: Oct. 28, 2022

(30) Foreign Application Priority Data
Aug. 17, 2022 (TW) 111208919

(51) Int. Cl. H01R 12/72; H01R 13/62; A61B 5/00; A61B 5/318; A61B 7/04; A61N 1/362; A61N 1/378

(52) U.S. Cl. CPC H01R 12/722; H01R 13/6205; A61B 5/0006; A61B 5/318; A61B 7/04; A61N 1/362; A61N 1/378; A61B 2560/0214

(57) ABSTRACT

A medical device assembly is provided, including a main body and a battery-charging module detachably connected to the main body. A plurality of first conductive pins and two metal parts are disposed on the main body. The metal parts have high magnetic permeability, and they are not permanent magnets. A plurality of second conductive pins and two magnets are disposed on the battery-charging module, wherein the magnets are disposed in locations corresponding to the metal parts.

### CROSS REFERENCE TO RELATED APPLICATIONS

This application claims priority of Taiwan Patent Application No. 111208919, filed on Aug. 17, 2022, the entirety of which is incorporated by reference herein.

### BACKGROUND OF THE INVENTION

### Field of the Invention

The present invention relates to a medical device assembly, and, in particular, to a medical device assembly that has a magnetic charging interface.

### Description of the Related Art

It is difficult to design a magnetic charging interface in a novel medical electronic device (e.g. artificial pacemaker, a wireless EKG sensor, or a wireless stethoscope) because magnets can interfere with medical electronic devices.

Hence, it is a challenge to design a medical device assembly that is provided with a magnetic charging interface yet without any magnets disposed in the main body.

### BRIEF SUMMARY OF THE INVENTION

An embodiment of the present invention provides a medical device assembly that includes a main body, a plurality of first conductive pins, two metal parts, a battery-charging module, a plurality of second conductive pins, and two magnets. The first conductive pins are disposed on the main body. The two metal parts are disposed on the main body and have high magnetic permeability, wherein the two metal parts are not permanent magnets.

The battery-charging module is detachably connected to the main body. The second conductive pins are disposed on the battery-charging module. The two magnets are disposed on the battery-charging module. When the battery-charging module is joined to the main body, the magnets and the metal parts attract each other, and the first conductive pins are electrically connected to the second conductive pins.

In some embodiments, the first conductive pins and the metal parts are exposed to the first side of the main body.

In some embodiments, the second conductive pins and the magnets are exposed to the second side of the battery-charging module.

In some embodiments, the medical device assembly further includes a first magnetic yoke disposed in the main body and connected between the two metal parts.

In some embodiments, the first magnetic yoke and the two metal parts form a U-shaped structure.

I In some embodiments, the medical device assembly further includes a circuit board disposed in the main body and located between the two metal parts, wherein the first conductive pins are disposed on the circuit board.

In some embodiments, the circuit board is affixed to the first magnetic yoke.

In some embodiments, the medical device assembly further includes a second magnetic yoke disposed in the battery-charging module and connected between the two magnets.

In some embodiments, the second magnetic yoke and the two magnets form a U-shaped structure.

In some embodiments, the main body may include an artificial pacemaker, a wireless EKG sensor, or a wireless stethoscope.

### BRIEF DESCRIPTION OF THE DRAWINGS

The present invention can be more fully understood by reading the subsequent detailed description and examples with references made to the accompanying drawings, wherein:

FIG. 1 is a schematic diagram of a medical device assembly 100, in accordance with an embodiment of the invention.

FIG. 2 is a schematic diagram of a medical device assembly 100, in accordance with another embodiment of the invention.

FIG. 3 is a schematic diagram of a medical device assembly 100, in accordance with another embodiment of the invention.

FIG. 4 is a perspective diagram of a medical device assembly 200, in accordance with another embodiment of the invention.

FIG. 5 is a perspective diagram of the main body 21 in FIG. 4.

FIG. 6 is an exploded diagram of the main body 21 in FIG. 5 with the bottom cover removed therefrom.

FIG. 7 is an exploded diagram of the battery-charging module 22 in FIG. 4.

FIG. 8 is another exploded diagram of the battery-charging module 22 in FIG. 4.

FIG. 9 is an exploded diagram of some components of the battery-charging module 22 in FIG. 7.

FIG. 10 is an enlarged cross-sectional view of the main body 21 and the battery-charging module 22 when connected to each other.

### DETAILED DESCRIPTION OF THE INVENTION

The making and using of the embodiments of the medical device assembly are discussed in detail below. It should be appreciated, however, that the embodiments provide many applicable inventive concepts that can be embodied in a wide variety of specific contexts. The specific embodiments discussed are merely illustrative of specific ways to make and use the embodiments, and do not limit the scope of the disclosure.

Unless defined otherwise, all technical and scientific terms used herein have the same meaning as commonly understood by one of ordinary skill in the art to which this invention belongs. It should be appreciated that each term, which is defined in a commonly used dictionary, should be interpreted as having a meaning conforming to the relative skills and the background or the context of the present disclosure, and should not be interpreted in an idealized or overly formal manner unless defined otherwise.

In the following detailed description of the preferred embodiments, reference is made to the accompanying drawings, and in which specific embodiments of which the invention may be practiced are shown by way of illustration. In this regard, directional terminology, such as “top,” “bottom,” “left,” “right,” “front,” “back,” etc., is used with reference to the orientation of the figures being described. The components of the present invention can be positioned in a number of different orientations. As such, the directional terminology is used for the purposes of illustration and is in no way limiting.

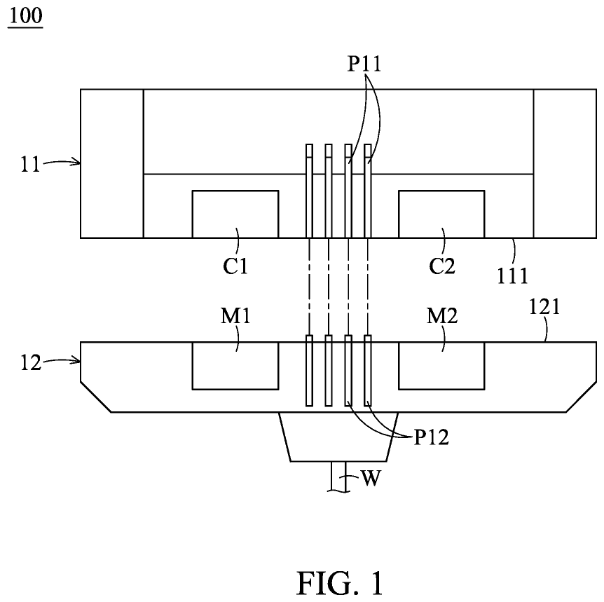

FIG. 1 is a schematic diagram of a medical device assembly 100, in accordance with an embodiment of the invention.

Referring to FIG. 1, an embodiment a medical device assembly 100 primarily comprises a main body 11 and a battery-charging module 12. The battery-charging module 12 is detachably connected to the main body 11, whereby the battery unit inside the main body 11 can be charged by the battery-charging module 12.

For example, the main body 11 may comprise an artificial pacemaker, a wireless EKG sensor, or a wireless stethoscope for measuring physiological signals or regulating the rhythm of the heart. As shown in FIG. 1, two metal parts C1 and C2 (e.g. iron blocks or iron sheets with high magnetic permeability) are disposed on a first side 111 of the main body 11.

Moreover, two magnets M1 and M2 are disposed on a second side 121 of the battery-charging module 12. It should be noted that the two magnets M1 and M2 are disposed in locations corresponding to the two metal parts C1 and C2, wherein the two metal parts C1 and C2 are not permanent magnets.

In this embodiment, a plurality of first conductive pins P11 are disposed in the main body 11, and a plurality of second conductive pins P12 are disposed in the battery-charging module 12. The first conductive pins P11 and the two metal parts C1 and C2 are exposed to the first side 111 of the main body 11. The second conductive pins P12 and the two magnets M1 and M2 are exposed to the second side 121 of the battery-charging module 12.

In some embodiments, the two metal parts C1 and C2 may be concealed in the main body 11, and the two magnets M1 and M2 may be concealed in the battery-charging module 12, which is not limited to the embodiments of the invention.

When charging the main body 11 by the battery-charging module 12, the second side 121 of the battery-charging module 12 is oriented toward to the first side 111 of the main body 11, whereby the two metal parts C1 and C2 and the two magnets M1 and M2 attract each other, and the first conductive pins P11 are electrically connected to the second conductive pins P12. The battery-charging module 12 can be electrically connected to a power supply or computer via the wire W, so that a battery unit (not shown) inside the main body 11 is charged by the battery-charging module 12 through the first and second conductive pins P11 and P12.

It should be noted that no magnet is disposed inside the main body 11 (e.g. artificial pacemaker, a wireless EKG sensor, or a wireless stethoscope). Hence, the main body 11 would not be interfered by any magnet when in use, thus facilitating safe and convenient usage of the medical device assembly 100.

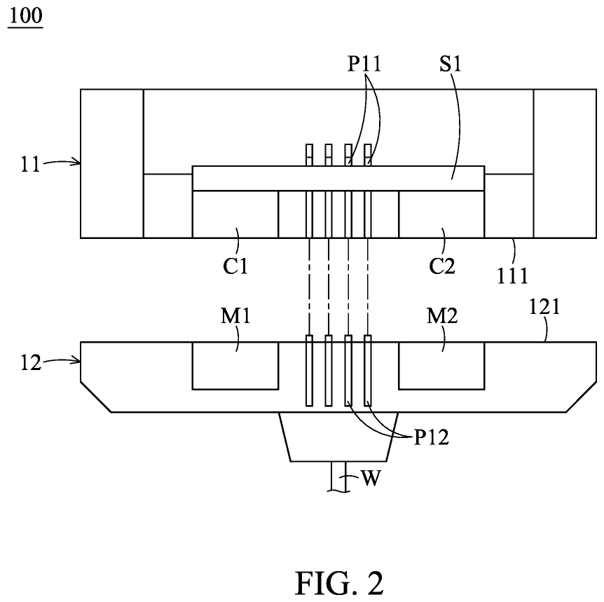

FIG. 2 is a schematic diagram of a medical device assembly 100, in accordance with another embodiment of the invention. FIG. 2 is different from FIG. 1 in that the medical device assembly 100 further comprises a first magnetic yoke S1 that is connected between the two metal parts C1 and C2, wherein the first magnetic yoke S1 and the two metal parts C1 and C2 form a U-shaped structure.

For example, the first magnetic yoke S1 may comprise a magnetic permeable metal sheet (e.g. iron sheet with high magnetic permeability). It should be noted that the magnetic field inside the medical device assembly 100 can be concentrated by the first magnetic yoke S1 connecting to the two metal parts C1 and C2 when the battery-charging module 12 is joined to the main body 11. Therefore, the magnetic attraction between the battery-charging module 12 and the main body 11 can be enhanced to achieve high reliability and safety of the medical device assembly 100.

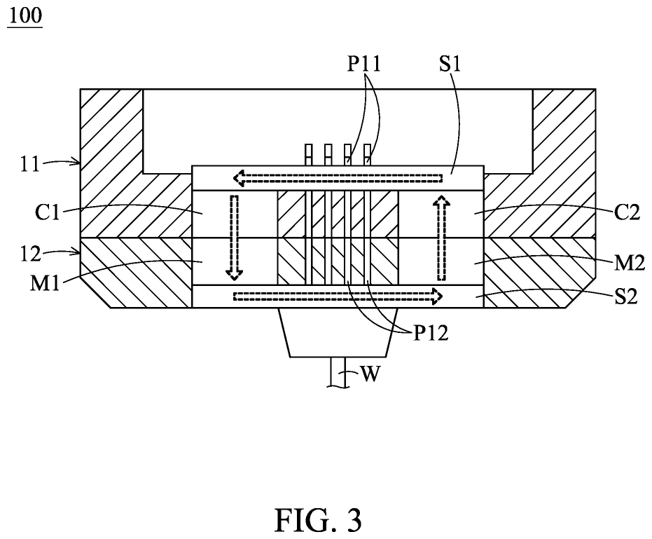

FIG. 3 is a schematic diagram of a medical device assembly 100, in accordance with another embodiment of the invention. FIG. 3 is different from FIG. 2 in that the medical device assembly 100 further comprises a second magnetic yoke S2 that is connected between the two magnets M1 and M2, wherein the second magnetic yoke S2 and the two magnets M1 and M2 form a U-shaped structure.

For example, the second magnetic yoke S2 may be comprise a magnetic permeable metal sheet (e.g. iron sheet with high magnetic permeability). When the battery-charging module 12 is joined to the main body 11, the magnetic field inside the medical device assembly 100 can be concentrated to form a closed magnetic path by the first magnetic yoke S1 connected between the two metal parts C1 and C2 and the second magnetic yoke S2 connected between the two magnets M1 and M2. Therefore, the magnetic attraction between the battery-charging module 12 and the main body 11 can be further enhanced to achieve high reliability and safety of the medical device assembly 100.

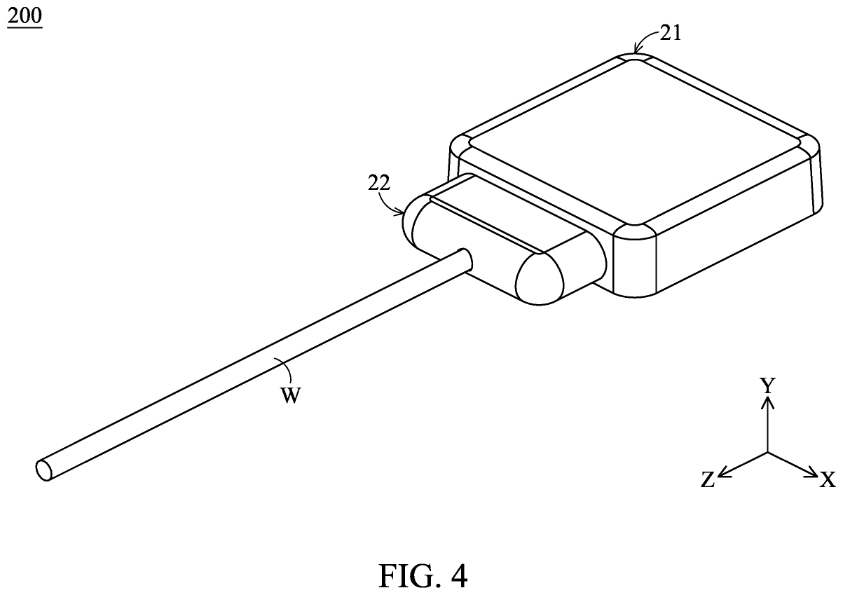

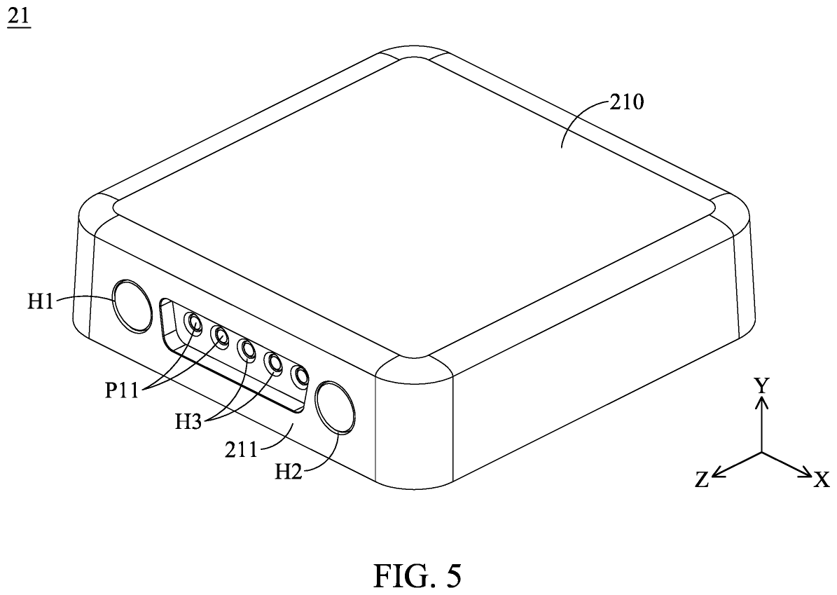

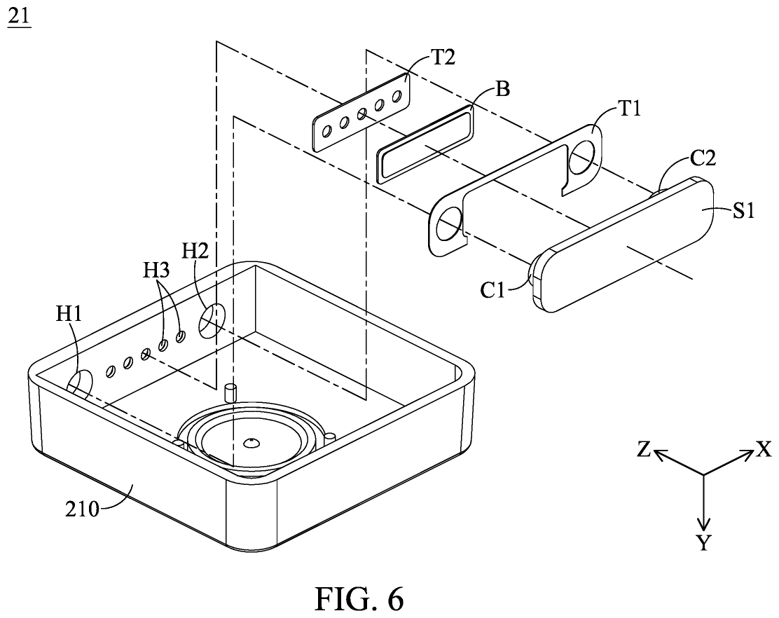

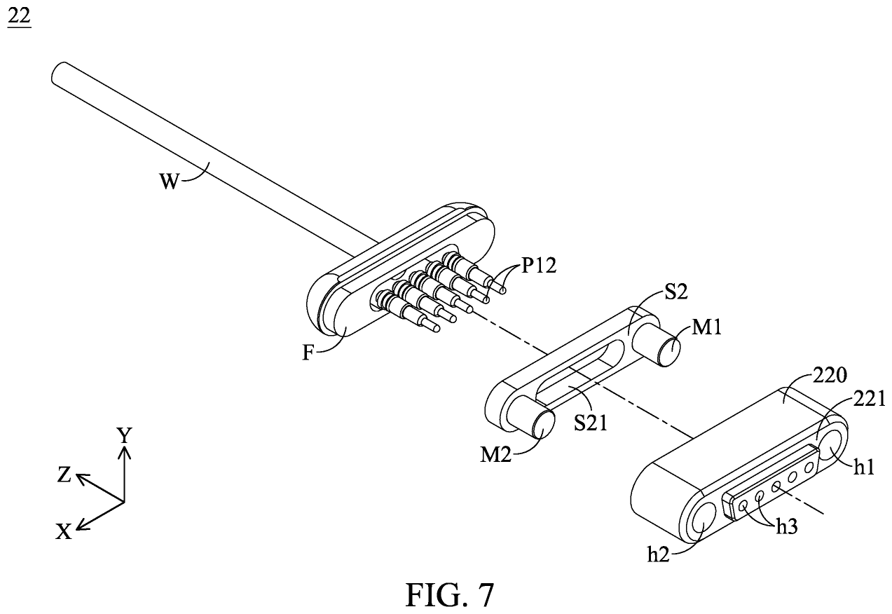

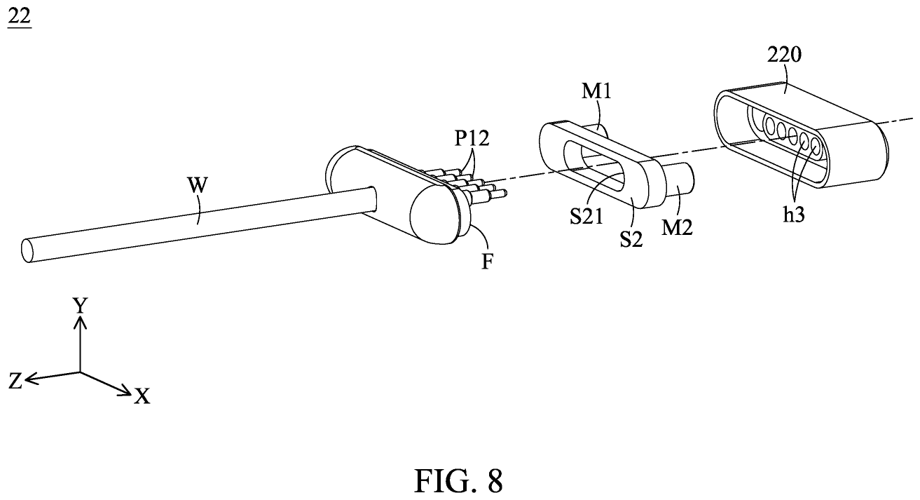

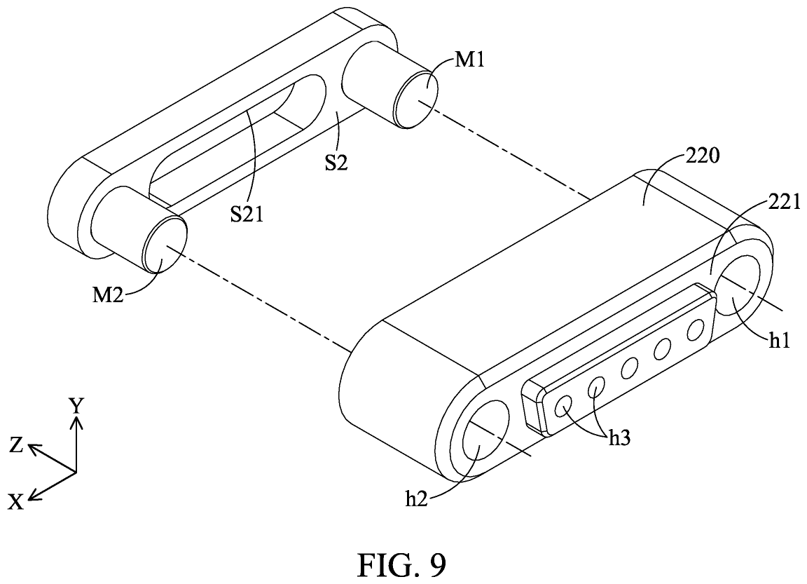

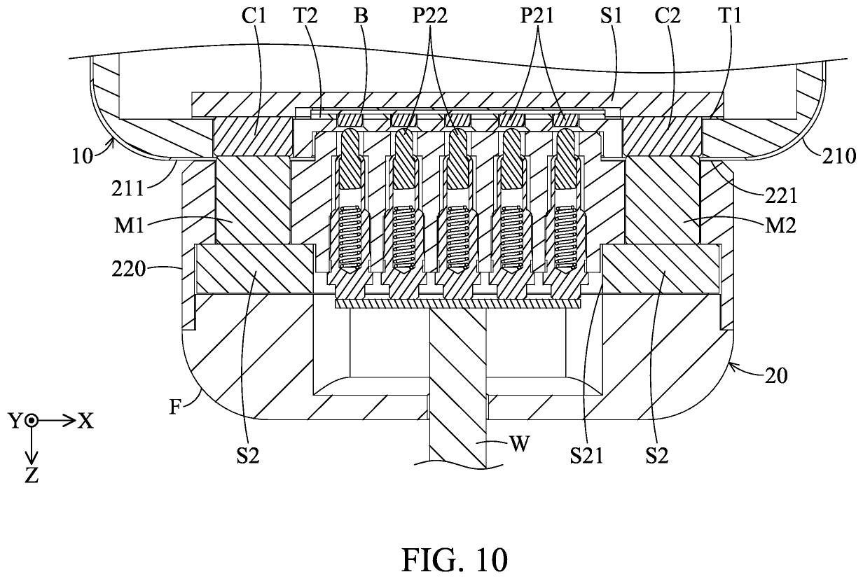

FIG. 4 is a perspective diagram of a medical device assembly 200, in accordance with another embodiment of the invention. FIG. 5 is a perspective diagram of the main body 21 in FIG. 4. FIG. 6 is an exploded diagram of the main body 21 in FIG. 5 with the bottom cover removed therefrom. FIG. 7 is an exploded diagram of the battery-charging module 22 in FIG. 4. FIG. 8 is another exploded diagram of the battery-charging module 22 in FIG. 4. FIG. 9 is an exploded diagram of some components of the battery-charging module 22 in FIG. 7. FIG. 10 is an enlarged cross-sectional view of the main body 21 and the battery-charging module 22 when connected to each other.

As shown in FIGS. 4-6, another embodiment of the medical device assembly 200 primarily comprises a main body 21 and a battery-charging module 22. Here, the main body 21 may comprise an artificial pacemaker, a wireless EKG sensor, or a wireless stethoscope for measuring physiological signals or regulating the rhythm of the heart.

In this embodiment, two metal parts C1 and C2 (e.g. iron blocks or iron sheets with high magnetic permeability) are disposed on a first side 211 of the main body 21. Moreover, two magnets M1 and M2 are disposed on a second side 221 of the battery-charging module 22. It should be noted that the two magnets M1 and M2 are disposed in locations corresponding to the two metal parts C1 and C2, and the two metal parts C1 and C2 are not permanent magnets.

The main body 21 has a housing 210, and several holes H1, H2 and H3 are formed on the housing 210. Specifically, the two metal parts C1 and C2 are exposed to the first side 211 of the main body 11 via the holes H1 and H2, and the first conductive pins P11 are exposed to the first side 211 of the main body 11 via the holes H3.

Referring FIG. 6, a first magnetic yoke S1, a first adhesion layer T1, a circuit board B, and a second adhesion layer T2 are provided in the housing 210. The first magnetic yoke S1 may comprise a magnetic permeable metal sheet (e.g. an iron sheet with high magnetic permeability), and the two metal parts C1 and C2 are affixed to the first magnetic yoke S1. One side of the circuit board B is affixed to the first magnetic yoke S1 by the first adhesion layer T1 (e.g. double-sided tape), and the circuit board B is located between the two metal parts C1 and C2 along the X axis.

The other side of the circuit board B is affixed to the inner surface of the housing 210 via the second adhesion layer T2. Hence, the circuit board B, the first magnetic yoke S1, and the two metal parts C1 and C2 can be stably received in the housing 210. In this embodiment, the first conductive pins P11 are disposed on the circuit board B and exposed to the first side 111 of the main body 11 through the holes H3 (FIGS. 5 and 10).

As shown in FIGS. 4 and 7-10, the battery-charging module 22 has a front cover 220 and a rear cover F. The front cover 220 and the rear cover F are connected to each other, thus forming a housing of the battery-charging module 22 to receive the two magnets M1 and M2 and the second magnetic yoke S2.

Specifically, several holes h1, h2, and h3 are formed on the front cover 220. The two magnets M1 and M2 are exposed to the second side 221 of the front cover 220 via the holes h1 and h2. The second conductive pins P12 extends through the hole S21 of the second magnetic yoke S2 into the holes h3 and are exposed to the second side 221 of the front cover 220.

Referring to FIG. 10, with the first magnetic yoke S1 connected between the two metal parts C1, C2 and the second magnetic yoke S2 connected between the two magnets M1, M2, the magnetic field inside the medical device assembly 100 can be concentrated to form a closed magnetic path when the battery-charging module 12 is joined to the main body 11. Therefore, the magnetic attraction and connection strength between the battery-charging module 12 and the main body 11 can be enhanced to achieve high reliability and safety of the medical device assembly 100. In this embodiment, the polar direction of the magnet M1 is opposite to the polar direction of the magnet M2.

In this embodiment, the two metal parts C1 and C2 are exposed to the first side 111 of the main body 11, and the two magnets M1 and M2 are exposed to the second side 121 of the battery-charging module 12. In some embodiments, however, the metal parts C1 and C2 may be concealed in the main body 11, and the magnets M1 and M2 may be concealed in the battery-charging module 12, which is not limited to the embodiments of the invention.

Although some embodiments of the present disclosure and their advantages have been described in detail, it should be understood that various changes, substitutions and alterations can be made herein without departing from the spirit and scope of the disclosure as defined by the appended claims. For example, it will be readily understood by those skilled in the art that many of the features, functions, processes, and materials described herein may be varied while remaining within the scope of the present disclosure. Moreover, the scope of the present application is not intended to be limited to the particular embodiments of the process, machine, manufacture, compositions of matter, means, methods and steps described in the specification. As one of ordinary skill in the art will readily appreciate from the disclosure of the present disclosure, processes, machines, manufacture, compositions of matter, means, methods, or steps, presently existing or later to be developed, that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps. Moreover, the scope of the appended claims should be accorded the broadest interpretation so as to encompass all such modifications and similar arrangements.

While the invention has been described by way of example and in terms of preferred embodiment, it should be understood that the invention is not limited thereto. On the contrary, it is intended to cover various modifications and similar arrangements (as would be apparent to those skilled in the art). Therefore, the scope of the appended claims should be accorded the broadest interpretation to encompass all such modifications and similar arrangements.

What is claimed is:

1. A medical device assembly, comprising:
a main body;
a plurality of first conductive pins, disposed on the main body;
two metal parts, disposed on the main body and having high magnetic permeability, wherein the two metal parts are not permanent magnets;
a battery-charging module, detachably connected to the main body;
a plurality of second conductive pins, disposed on the battery-charging module; and
two magnets, disposed on the battery-charging module, wherein when the battery-charging module is joined to the main body, the magnets and the metal parts attract each other, and the first conductive pins are electrically connected to the second conductive pins.

2. The medical device assembly as claimed in claim 1, wherein the first conductive pins and the metal parts are exposed to a first side of the main body.

3. The medical device assembly as claimed in claim 2, wherein the second conductive pins and the magnets are exposed to a second side of the battery-charging module.

4. The medical device assembly as claimed in claim 1, further comprising a first magnetic yoke disposed in the main body and connected between the two metal parts.

5. The medical device assembly as claimed in claim 4, wherein the first magnetic yoke and the two metal parts form a U-shaped structure.

6. The medical device assembly as claimed in claim 4, further comprising a circuit board disposed in the main body and located between the two metal parts, wherein the first conductive pins are disposed on the circuit board.

7. The medical device assembly as claimed in claim 6, wherein the circuit board is affixed to the first magnetic yoke.

8. The medical device assembly as claimed in claim 4, further comprising a second magnetic yoke disposed in the battery-charging module and connected between the two magnets.

9. The medical device assembly as claimed in claim 8, wherein the second magnetic yoke and the two magnets form a U-shaped structure.

10. The medical device assembly as claimed in claim 1, wherein the main body comprises an artificial pacemaker, a wireless EKG sensor, or a wireless stethoscope.
